(19) United States
(12) Patent Application Publication
(10) Pub. No.: US 2026/0315273 A1
(43) Pub. Date: Oct. 15, 2026

(54) EYE TRACKING SYSTEM FOR OCULAR SURGERY

(71) Applicant: Alcon Inc., Fribourg (CH)

(72) Inventor: Steven T. CHARLES, Memphis, TN (US)

(21) Appl. No.: 19/629,323

(22) Filed: Mar. 26, 2026

Related U.S. Application Data
(60) Provisional application No. 63/789,187, filed on Apr. 15, 2025.

(51) Int. Cl. A61F 9/007; A61B 3/113; A61B 5/11; A61B 34/30; A61B 90/14; A61B 17/00; A61B 90/00; A61B 90/50

(52) U.S. Cl. CPC A61F 9/007; A61B 3/113; A61B 5/1114; A61B 34/30; A61B 90/14; A61B 2017/00057; A61B 2017/00123; A61B 2090/3735; A61B 2090/506; A61B 2562/0219

(57) ABSTRACT

The present disclosure relates to an eye tracking system for a surgical system for ophthalmic microsurgical procedures. In certain embodiments, a surgical system includes a robotic manipulator and a surgical tool assembly connected to the robotic manipulator. The surgical tool assembly includes an actuatable surgical tool, and the robotic manipulator is configured to position the surgical tool assembly above an eye of a patient. The surgical system further includes an eye tracking system comprising one or more Time of Flight (ToF) sensors positioned above the eye, the one or more ToF sensors configured to acquire pose information of the eye. The surgical system further includes a controller communicatively connected to the surgical tool and to the eye tracking system, the controller configured to disable operation of the surgical tool responsive to excessive movement of the eye that is indicated by the pose information.

### CROSS-REFERENCE TO RELATED APPLICATIONS

This application claims the benefit of priority of U.S. Provisional Patent Application Serial No. 63/789,187 titled " EYE TRACKING SYSTEM FOR OCULAR SURGERY," filed on April 15, 2025, which is hereby incorporated by reference in its entirety as though fully and completely set forth herein.

### INTRODUCTION

Ophthalmic microsurgical procedures, such as vitreoretinal microsurgeries, cataract surgeries, glaucoma surgeries, and corneal surgeries, are among the most challenging ophthalmic surgical procedures. During ophthalmic microsurgical procedures, surgeons monitor patients’ eyes through surgical microscopes and manipulate micro-instruments to minimize trauma to the anatomical structures of the eye. The surgeons are expected to perform precise micron-scale maneuvers while applying diminutive forces to ocular tissues, often beyond the natural human levels of sensory perception. Thus, the performance of ophthalmic microsurgical procedures is inherently restricted by human sensory and motor limitations, surgeon fatigue and hand tremor, imprecise instrumentation, fine feature sizes, limited manipulation room within the ocular space, and occasionally poor visualization of the interior of the eye.

In addition to the above limitations, serious complications may also be caused by involuntary movement of the patient’s head and/or eye. These factors may contribute to a variety of surgical complications including retinal or lens damage, capsular rupture, hemorrhage, corneal abrasions or perforations, compromised suture placement, retinal tear or detachment, lens dislocation, and so forth, many of which can develop into potentially irreversible damage and visual impairment.

Robotically-assisted surgical devices are becoming increasingly common to assist surgeons in minimally-invasive ophthalmic surgical procedures. In many cases, these surgical procedures are performed while the patient is awake, without the use of anesthesia and neuromuscular blockades. As a result, robotic surgical devices remain vulnerable to high risk of complications from patient eye movement during surgery. Further, the relatively small size of surgical instruments that are used for ophthalmic surgical procedures and other similarly precise procedures prevents the surgical instruments from contributing effectively to the immobilization of the eye.

### SUMMARY

The present disclosure relates to robotic manipulation systems for surgical procedures, and more particularly, to an eye tracking system that improves safety and efficiency of the robotic manipulation systems for ophthalmic microsurgical procedures.

In certain embodiments, a surgical system includes a robotic manipulator and a surgical tool assembly connected to the robotic manipulator. The surgical tool assembly includes an actuatable surgical tool, and the robotic manipulator is configured to position the surgical tool assembly above an eye of a patient. The surgical system further includes an eye tracking system comprising one or more Time of Flight (ToF) sensors positioned above the eye, the one or more ToF sensors configured to acquire pose information of the eye. The surgical system further includes a controller communicatively connected to the surgical tool and to the eye tracking system, the controller configured to disable operation of the surgical tool responsive to excessive movement of the eye that is indicated by the pose information.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1 illustrates a schematic view of an exemplary surgical system, according to one or more embodiments.

FIG. 2A illustrates an example perspective view of a surgical tool assembly and eye tracking system of the surgical system of FIG. 1, according to one or more embodiments.

FIG. 2B illustrates an example schematic top-down view of the surgical tool assembly and eye tracking system of FIG. 2A, according to one or more embodiments.

FIG. 2C illustrates an example schematic bottom-up view of the surgical tool assembly and eye tracking system of FIG. 2A, according to one or more embodiments.

FIG. 3 illustrates an example perspective view of a surgical tool assembly of the surgical system of FIG. 1, according to one or more embodiments.

FIG. 4 illustrates an example method of operating a surgical tool, according to one or more embodiments.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

In the following description, details are set forth by way of example to facilitate an understanding of the disclosed subject matter. It should be apparent to a person of ordinary skill in the field, however, that the disclosed implementations are exemplary and not exhaustive of all possible implementations. Thus, it should be understood that reference to the described examples is not intended to limit the scope of the disclosure. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one implementation may be combined with the features, components, and/or steps described with respect to other implementations of the present disclosure.

Note that, as described herein, a distal end or portion of a component refers to the end or the portion that is closer to a patient’s body during use thereof. On the other hand, a proximal end or portion of the component refers to the end or the portion that is distanced further away from the patient’s body.

As used herein, the term “about” may refer to a +/-10% variation from the nominal value. It is to be understood that such a variation can be included in any value provided herein.

Embodiments of the present disclosure relate to an eye tracking system that improves safety and efficiency of robotic manipulation systems for ophthalmic microsurgical procedures. In certain embodiments, the eye tracking system of a surgical system includes one or more Time of Flight (ToF) sensors that provide high-resolution, high-precision tracking of the patient’s eye, allowing any movement of the patient during the surgical procedure to be rapidly and precisely identified. The one or more TOF sensors may be used in conjunction with one or more additional sensors that provide imaging of the eye, such as stereo machine vision sensors, an optical coherence tomography (OCT) sensor, a polarization sensor, and so forth. With this improved tracking capability, a surgical tool (whether controlled robotically or by a human operator) can be disabled or otherwise controlled to mitigate or eliminate risk of unnecessary damage to a patient’s eye resulting from involuntary movement. In some embodiments, the controller of the eye tracking system detects excessive eye movement using various techniques and responsively disables the surgical tool or modifies its operation. In some embodiments, disabling the surgical tool comprises transmitting a control signal to the surgical tool, removing the operative power therefrom, retracting the surgical tool or a component away from the eye, and so forth. Accordingly, the surgical system improves the safety and efficiency of ophthalmic microsurgical procedures, whether performed by human operator or by robotic means.

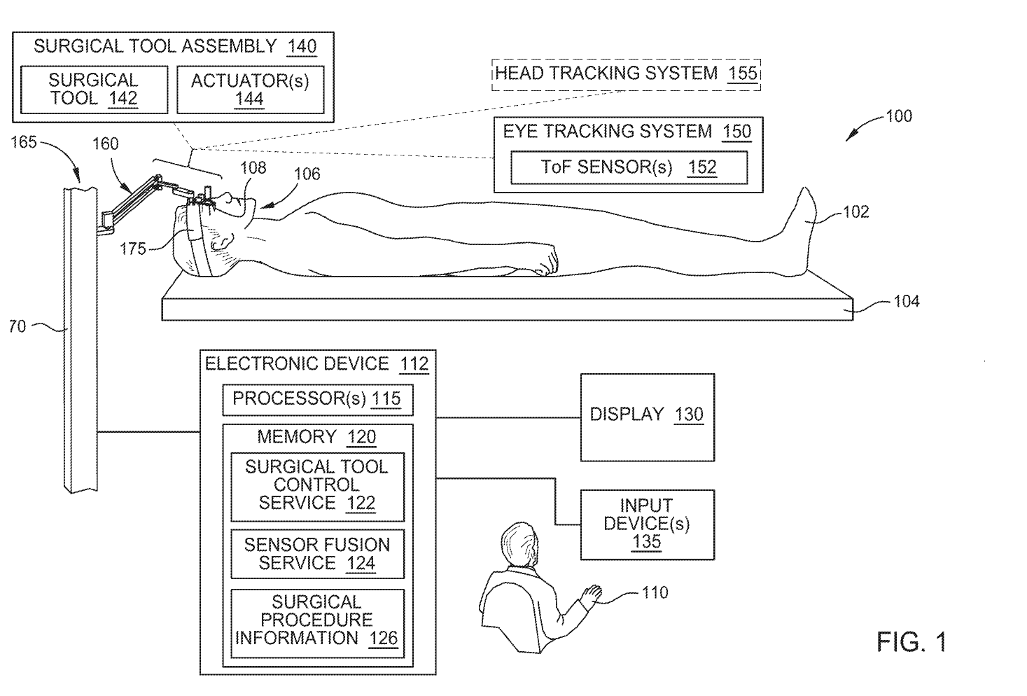

FIG. 1 illustrates a schematic view of an exemplary surgical system 100, according to one or more embodiments. In some embodiments, the surgical system 100 is implemented as a robotic surgical system capable of autonomously operating one or more surgical tools 142 according to one or more algorithm(s) to perform a surgical procedure on a patient 102. In other embodiments, a human operator 110 may perform the surgical procedure using functionality provided by the surgical system 100, such as eye tracking and/or operating the one or more surgical tools 142 according to input(s) provided by the human operator 110.

The surgical system 100 comprises an electronic device 112 that is communicatively connected with a surgical tool assembly 140, an eye tracking system 150, and optionally a head tracking system 155. In some embodiments, the electronic device 112 is further connected with a display 130 and one or more input devices 135 to support communication with the human operator 110.

The surgical tool assembly 140 comprises a surgical tool 142 that is an actuatable surgical tool connected to one or more actuators 144. The electronic device 112 controls the surgical tool 142 and the one or more actuators 144 to position and operate the surgical tool 142 during a surgical procedure. As used herein, an “electronic device” generally refers to any device having electronic circuitry that provides a processing or computing capability, and that implements logic and/or executes program code to perform various operations that collectively define the functionality of the electronic device. The functionality of the electronic device includes a communicative capability with one or more other electronic devices, e.g., when connected to a same network (e.g., a Local Area Network or a Wide Area Network). An electronic device may be implemented with any suitable form factor, whether relatively static in nature (e.g., mainframe, computer terminal, server, kiosk, workstation,) or mobile (e.g., laptop computer, tablet, handheld, smart phone, wearable device). In some embodiments, the electronic device 112 may be fully or partially implemented in a surgical console. The communicative capability between electronic devices may be achieved using any suitable techniques, such as conductive cabling, wireless transmission, optical transmission, and so forth.

The electronic device 112 comprises one or more processors 115 and a memory 120. The one or more processors 115 represent any electronic circuitry, including, but not limited to one or a combination of microprocessors, microcontrollers, application-specific integrated circuits (ASIC), application-specific instruction set processors (ASIP), and/or state machines, that is communicatively coupled to the memory and controls the operation of the system. In some aspects, the electronic circuitry is configured to perform any of the functions described herein. Further, the one or more processors are not limited to a single processing device and may encompass multiple processing devices.

The one or more processors 115 may include other hardware that operates software to control and process information. In some aspects, the one or more processors 115 execute software stored in the memory to perform any of the functions described herein. The one or more processors 115 control the operation and administration of the electronic device by processing information (e.g., information received from input devices and/or communicatively coupled electronic devices).

The memory 120 may store, either permanently or temporarily, data, operational software, or other information for the one or more processors. The memory 120 may include any one or a combination of volatile or non-volatile local or remote devices suitable for storing information. For example, the memory 120 may include random-access memory (RAM), read-only memory (ROM), magnetic storage devices, optical storage devices, or any other suitable information storage device or a combination of these devices. The software represents any suitable set of instructions, logic, or code embodied in a computer-readable storage medium. For example, the software may be embodied in the memory, a disk, a CD, or a flash drive. In particular embodiments, the software may include an application executable by the one or more processors to perform one or more of the functions described herein.

As shown, the memory 120 includes a surgical tool control service 122, a sensor fusion service 124, and surgical procedure information 126. The functionality of the surgical tool control service 122 (which may be in combination with the functionality of the sensor fusion service 124) may be referred to herein as a “controller”.

The surgical procedure information 126 includes information about the surgical procedure to be performed using the surgical system 100. For example, the surgical procedure information 126 may describe a type of the surgical procedure and one or more values corresponding to one or more parameters for the surgical procedure.

The surgical tool control service 122 controls the operation of the surgical tool 142 and the one or more actuators 144 to perform the surgical procedure according to the surgical procedure information 126. In some embodiments, the surgical tool control service 122 controls delivery of operative power to the surgical tool 142 and/or to the one or more actuators 144, e.g., selectively applying electrical, optical, hydraulic, and/or pneumatic power. In some embodiments, the surgical tool control service 122 generates control signals for the surgical tool 142 and the one or more actuators 144 that are based at least on the surgical procedure information 126, information about the surgical tool 142, and/or information about the patient 102. In one example, the surgical tool control service 122 receives an identifier that indicates a type of the surgical tool 142 and/or a unique identity (e.g., a serial number), and generates the control signals based on operational characteristics and/or previous usage of the surgical tool 142. In another example, the surgical tool control service 122 receives biometric information about the patient 102 and generates the control signals based thereon.

As will be discussed in greater detail below, the eye tracking system 150 and/or other components of the surgical system 100 capture imagery of the patient’s head 106 and/or eye 108 during the surgical procedure, and the surgical tool control service 122 generates the control signals based on the imagery. In some embodiments, the eye tracking system 150 comprises one or more Time of Flight (ToF) sensors 152 that provide high-resolution, high-precision tracking of the patient’s eye 108, allowing any movement of the patient 102 during the surgical procedure to be rapidly and precisely identified. The eye tracking system 150 may include one or more other sensors that provide imaging of the eye 108, such as stereo visual sensors, an OCT sensor, a polarization sensor, and so forth. In some embodiments, the surgical tool control service 122 receives sensor signals from various sensors of the surgical system 100 (e.g., representing the captured imagery) and determines pose information of the patient’s eye 108 and/or head 106. As used herein, a “pose” encompasses a position and an orientation in any suitable dimensional space (e.g., 2D or 3D). Based on the pose information, the surgical tool control service 122 dynamically orients and operates the surgical tool 142 to safely and efficiently accomplish the surgical procedure. For example, in some embodiments, the surgical tool control service 122 determines whether the pose information of the eye 108 and/or head 106 indicates excessive movement of the eye 108, and disables operation of the surgical tool 142 responsive to excessive movement. Other actions by the surgical tool control service 122 are also contemplated, such as displacing the surgical tool 142 away from the eye 108, manipulating the movement of the surgical tool 142 to track the movement of the eye 108 (e.g., closed-loop position error correction), changing the operational state or parameters of the surgical tool 142, communicating warnings to the patient 102 and/or the human operator 110 about the movement of the eye 108, and so forth.

While anesthesia and neuromuscular blockades can be effective to prevent patient movement during surgical procedures, these approaches can potentially disrupt utilization of the surgical tool 142 and in some cases can lead to surgical tool-induced damage to the patient’s eye 108. Using the techniques described herein, the eye tracking system 150 rapidly and precisely identifies movement of the patient’s eye 108 during the surgical procedure, allowing the surgical tool 142 to be controlled in a way that greatly reduces or eliminates the risks associated with the patient’s movement, without requiring administration of anesthesia or neuromuscular blockades.

During the surgical procedure, the surgical tool assembly 140 is positioned above the eye 108 of the patient 102, who is shown in FIG. 1 as lying in a surgical position on an operating table 104. The surgical tool assembly 140 comprises a surgical tool 142 that is actuatable using one or more actuators 144. The surgical tool assembly 140 is at least partially supported over the patient’s eye 108 by a support system 165, which comprises a robotic manipulator 160. In some embodiments, and as shown, the robotic manipulator 160 comprises an articulating arm 162 that is supported by a base 170, such as a support post, or that extends from another surgical device or a ceiling of an operating room. In other embodiments, the robotic manipulator 160 comprises a parallel kinematic manipulator or a hybrid serial-parallel manipulator.

The surgical tool 142 includes any suitable surgical device or apparatus for ophthalmic microsurgical procedures. For example, the surgical tool 142 may encompass actuated devices such as vitreous cutters, fragmenters, phacoemulsification probes, etc. as well as non-actuated devices such as scissors, forceps, cannulas, picks, manipulators, choppers, etc. In certain embodiments, the surgical tool 142 is configured to perform surgical maneuvers, such as phacoemulsification, irrigation/aspiration, tissue/lens manipulation, membrane peeling, segmentation, delamination of epiretinal membranes, retinal incisions, subretinal injections, fluid-air exchange, posterior capsulotomy, scleral buckling, aspiration of vitreous hemorrhage, intraocular lens manipulation, endophotocoagulations, or the like.

In some embodiments, the support system 165 is configured to mount to the patient’s head 106 and support at least the surgical tool assembly 140. In some embodiments, the support system 165 further comprises a forehead support 175 that mounts to (e.g., rests on) the patient’s head 106. The articulating arm 162 employs a passive four-bar parallelogram mechanism, and in some embodiments is a counterbalancing articulating arm that is counterbalanced by an air spring or constant force spring that alleviates pressure on the patient’s head 106 that is caused by the weight of the surgical tool assembly 140 and the forehead support 175.

In other embodiments, the support system 165 need not be connected to the patient’s head 106 through the forehead support 175. For example, the surgical system 100 may comprise a head tracking system 155 that monitors a pose of the patient’s head 106 during the surgical procedure. In some embodiments, the head tracking system 155 comprises an inertial measurement unit (IMU) that is removably connected to the patient’s head 106, e.g., using a temporary adhesive or a forehead-worn item. The IMU may include one or more accelerometers, gyroscopes, and/or magnetometers that acquire inertial information of the patient’s head 106. In other embodiments, the head tracking system 155 may include one or more fiducial markers that are removably connected to the patient’s head 106 and detected using one or more visual sensors.

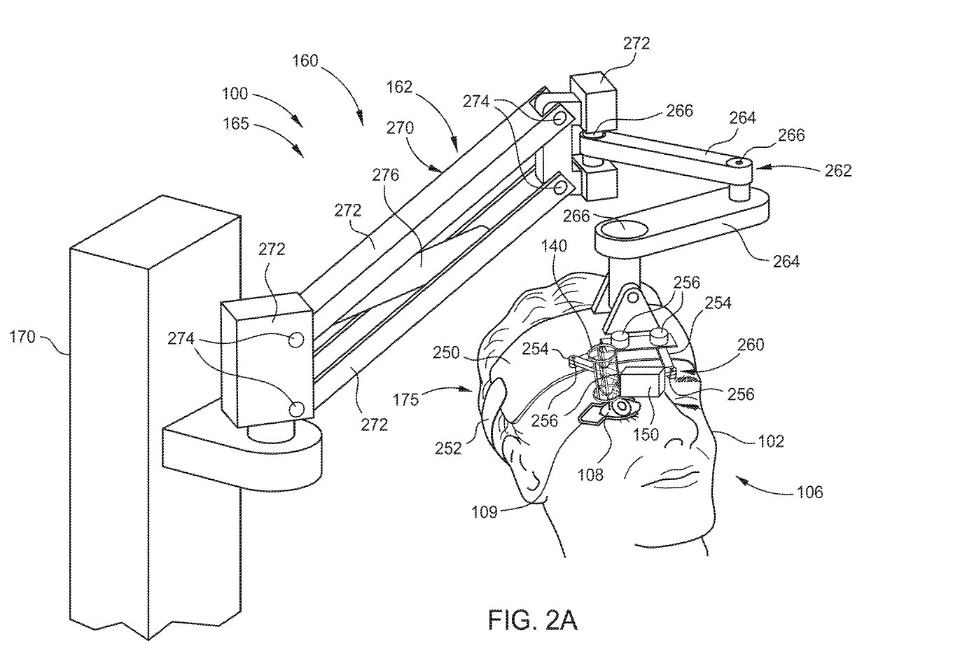

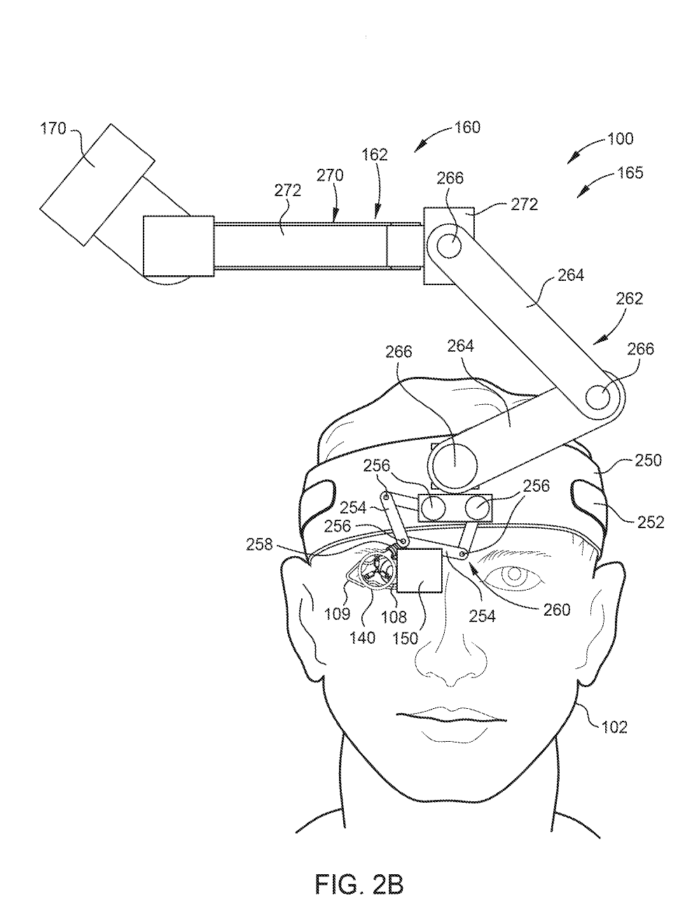

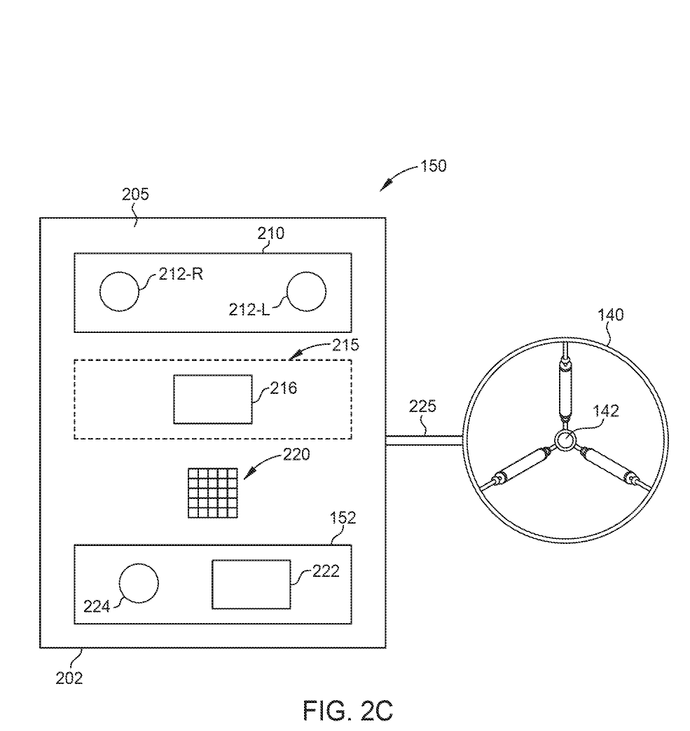

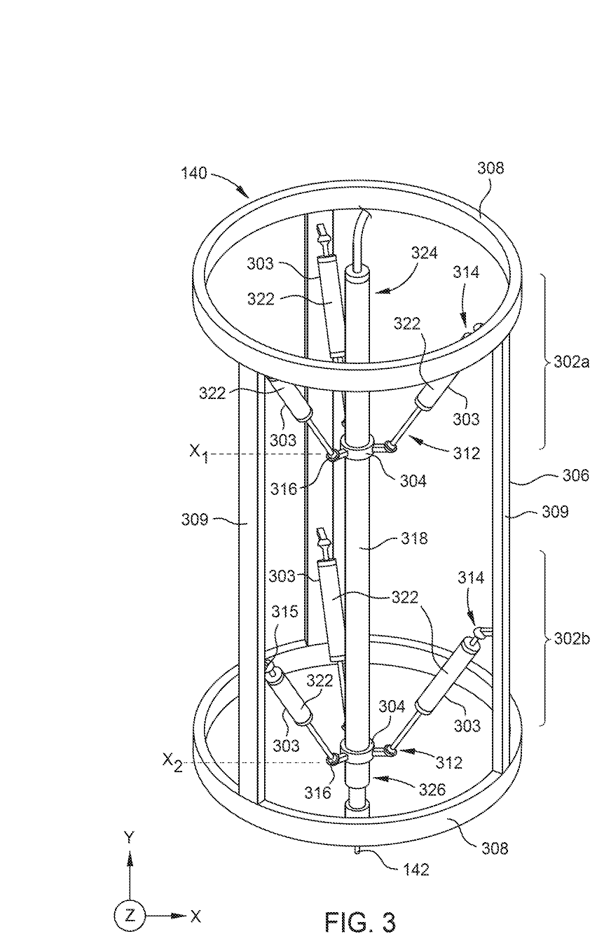

By mounting the surgical tool assembly 140 to the patient’s head 106, the risks associated with uncontrolled patient head movement during the surgical procedure can be greatly reduced or eliminated. Further details regarding the surgical tool assembly 140, the forehead support 175, and the articulating arm 162 are provided below with reference to FIGS. 2A, 2B, and 3.

During the surgical procedure, the eye tracking system 150 is also positioned above the eye 108 of the patient 102. In some embodiments, the eye tracking system 150 is connected to the support system 165, e.g., connected to the articulating arm 162. The eye tracking system 150 may be rigidly connected to the surgical tool assembly 140 or may be movable relative thereto. In other embodiments, the eye tracking system 150 is supported independently of the support system 165. For example, the eye tracking system 150 may be connected to another articulating arm, to a surgical console, and so forth.

The eye tracking system 150 comprises the one or more Time of Flight (ToF) sensors 152 that acquire pose information of the eye 108. In some embodiments, the one or more ToF sensors 152 comprises a plurality of ToF sensors 152, which may be operated in combination to acquire the pose information of the eye 108. In some embodiments, the plurality of ToF sensors 152 may be selectively operated to acquire the pose information of the eye 108. For example, a first ToF sensor 152 may be operated responsive to determining that the eye 108 is fully or partially occluded in the field of view of a second ToF sensor 152.

The ToF sensor(s) 152 may use any suitable ToF techniques, such as direct ToF and indirect ToF. Generally, direct ToF measures a length of time between emission of a pulse of light and detection of the reflected light. A ToF sensor 152 using direct ToF may include a plurality of single photon avalanche diode (SPAD) pixels, and processing circuitry such as avalanche photodiodes that use avalanche multiplication to generate many electrons from a single incident photon. Thus, a ToF sensor 152 using direct ToF can detect very faint light and is suitable for measuring at greater distances.

Indirect ToF typically operates by transmitting a periodic light signal (e.g., pulsed or continuous wave), receiving reflected light, and measuring distance through phase shifts in the accumulated electric charges that are generated by the reflected light. An indirect ToF sensor 152 may include a plurality of complementary metal-oxide-semiconductor (CMOS) image sensor pixels, which tends to have a less complex implementation and lower power consumption than a direct ToF-based ToF sensor 152. Thus, an indirect ToF sensor 152 is well-suited for high-speed, high-resolution 3D sensing at near- to mid-range, typically capable of achieving frame rates of 60 fps or greater at ranges of 5 meters or greater.

The eye tracking system 150 may further include other eye imaging functionality to support the surgical procedure. In some embodiments, the eye tracking system 150 further comprises one or more of the following sensors: two (e.g., stereo) visual sensors, an OCT sensor, and a polarization sensor.

The two visual sensors may have any suitable implementation that collectively provide stereo imaging of the eye 108, such as charge-coupled device (CCD) sensors or CMOS sensors.

The OCT sensor generally transmits near-infrared light, and the reflected light is combined with reference light propagating within the OCT sensor. The interference of the reflected light and reference light provides a one-dimensional signal in the depth direction. The OCT sensor may use any suitable techniques, such as spectral domain OCT (SD-OCT) and swept source OCT (SS-OCT). A SD-OCT based sensor typically includes a broadband wavelength light source, a beamsplitter that disperses the light onto a sample path and a reference path, a reference mirror along the reference path, and a linear image sensor that receives (through a grating) the reflected light from the sample and the reference light. A SS-OCT based sensor typically includes a wavelength-swept light source, a beam splitter and reference mirror, and a differential detector that receives the reflected light from the sample and the reference light.

The polarization sensor generally includes a polarizer array layer above a plurality of photosensors (e.g., photodiodes). Each pixel of the polarization sensor may be overlaid by one or more polarizers (e.g., a plurality of polarizers with different polarization angles) that allow the calculation of degree and/or direction of polarization of the received light.

In some embodiments, the sensor fusion service 124 performs a sensor fusion operation using sensor signals received from at least one of: the one or more ToF sensors 152, the two visual sensors, the OCT sensor, and the polarization sensor. The surgical tool control service 122 detects, from the sensor fusion operation, any excessive movement of the eye 108. In some embodiments where the one or more ToF sensors 152 comprises a plurality of ToF sensors 152, the sensor fusion operation may be performed on the sensor signals from the plurality of ToF sensors 152.

The sensor fusion service 124 may use any suitable technique(s), such as data-level fusion, feature-level fusion, decision-level fusion, Kalman filtering, particle filtering, machine learning, and so forth. In some embodiments, the surgical tool control service 122 implements a Kalman filter to perform the sensor fusion operation. A Kalman filter generally uses a model of the system and measurements of the various sensor signals to estimate a state of the system, which also accounts for uncertainty in the measurements.

As described above, using the one or more ToF sensors 152 of the eye tracking system 150, movement of the patient’s eye 108 may be rapidly and precisely identified during surgical procedures. This allows the surgical tool 142 to be controlled in a way that greatly reduces or eliminates the risks associated with the patient’s movement. In some embodiments, the surgical system 100 is implemented as a robotic surgical system that autonomously operates the surgical tool 142 to perform the surgical procedure. In other embodiments, the human operator 110 may manually operate the surgical tool 142.

In yet other embodiments, the human operator 110 may remotely operate the surgical tool 142 using the display 130 and/or the one or more input devices 135. In some embodiments, the one or more input devices 135 comprises an operator device that is communicatively connected to the surgical tool control service 122. Responsive to manipulation of the operator device by the human operator 110, the surgical tool control service 122 receives operator pose information from the operator device and controls the one or more actuators 144 of the surgical tool assembly 140 based on the operator pose information.

The surgical tool 142 may include an end effector having one or more actuators for enabling direct manipulation of the end effector secured thereto. In some embodiments, the end effector has a six degrees of freedom (6-DOF) force/torque sensor (i.e., a transducer) incorporated therein to facilitate force feedback and force control by the surgical system 100. In still further embodiments, the surgical tool 142 is a device holder or sleeve configured to secure another device or tool to the surgical tool assembly 140, and may include a radio frequency identification (RFID) or quick response (QR) barcode sensor in communication therewith to communicate tool weight (facilitating a weightless tool), moment arms (facilitating center of gravity compensation as an orientation of the surgical tool 142 changes in space), and tool length and offsets to the electronic device 112. Generally, tool actuation pneumatic or hydraulic connections, fiber optic connections, aspiration and/or injection connections, and an uninterruptible power supply connection may be incorporated into the surgical tool 142 or may bypass the surgical tool 142 via service loops.

In some embodiments, the operator device may be a controlling system and the surgical tool assembly 140 may be a controlled system. For example, the operator device may have any suitable type that is characterized by 6-DOF or 7-DOF and that includes an operator interface. In certain embodiments, the operator device includes a 6-DOF or 7-DOF haptic interface with low inertia and friction. One such example of a suitable operator device with a haptic interface is the Freedom6S haptic device available from MPB Technologies, Inc.

In certain embodiments, the operator device includes a haptic interface modeled to match (e.g., resemble) the surgical tool assembly 140. For example, the operator device may have a structure substantially similar to that of the surgical tool assembly 140, described in greater detail below. When the human operator 110 manipulates the operator device, the operator device generates a plurality of signals that are transmitted between the operator device, the electronic device 112, and the surgical tool assembly 140. The surgical tool assembly 140 controls the manipulation and/or operation of the surgical tool 142 directly or indirectly coupled thereto to perform an ophthalmic surgical procedure.

FIG. 2A illustrates an example perspective view of the surgical tool assembly 140 and eye tracking system 150 of the surgical system of FIG. 1, according to one or more embodiments. FIG. 2B illustrates an example schematic top-down view of the surgical tool assembly 140 and eye tracking system 150 of FIG. 2A, according to one or more embodiments.

As shown, the surgical tool assembly 140 is coupled to the support system 165. In certain aspects, the support system 165 aids in supporting the surgical tool assembly 140 in an upright and secured (e.g., fixed) position over the patient’s eye 108, which is held open by a speculum 109, to prevent relative movement between the patient’s head and the surgical tool assembly 140. For example, when the surgical tool assembly 140 is attached to the support system 165 and mounted on the patient’s head 106, the surgical tool assembly 140 will move with the patient’s head 106, thereby eliminating, or at least reducing, the need for general anesthesia and neuromuscular blockade. Anesthesia and neuromuscular blockades are typically used to prevent patient movement during surgical procedures, which can disrupt utilization of the surgical tool 140 and/or lead to surgical instrument-induced damage of the patient’s eye 108. Thus, the risks associated with involuntary movement of the patient may be greatly reduced or eliminated by utilizing the surgical tool assembly 140 and the support system 165. In certain aspects, the support system 165 further reduces or eliminates any pressure against the patient’s head 106 caused by the weight of the surgical tool assembly 140 by employing a counterbalancing mechanism. Accordingly, the patient 102 will not feel the weight of the surgical tool assembly 140, but may still feel the inertia, which will slow down and discourage patient movement.

The support system 165 generally includes the forehead support 175, the articulating arm 162, and base 170. The forehead support 175 comprises a sterilizable or disposable U-shaped forehead pad 250 configured to contact and rest on the patient’s forehead and temples. In order to improve comfort for the patent 102, an underside or patient-facing side of the forehead support 175 includes a surface padded with, for example, a viscoelastic material, such as dense memory foam. In certain embodiments, the forehead support 175 is secured to the patient’s head utilizing a broad and adjustable head strap 252, which may be fastened via any suitable fastening mechanism. In certain embodiments, the head strap 252 is adjustably fastened via a hook and loop fastener such as, for example, Velcro®, to enable a customized fit with respect to the patient. By attaching the surgical tool assembly 140 to the patient’s head 106 via the forehead support 175, patient head rotation relative to the surgical tool assembly 140 is virtually eliminated.

The surgical tool assembly 140 is attached to the forehead support 175 via an adjustable attachment 260 to accommodate different patient anatomical characteristics (e.g., head geometries) and enable lateral positioning of the surgical tool assembly 140 over the desired patient’s eye 108. In certain embodiments, the attachment 260 includes an adjustable arm, such as a single serial articulating arm or two parallel articulating arms, medially attached to the forehead support 175 to facilitate positioning of the surgical tool assembly 140 over either of the patient’s eyes. In the embodiment of FIGS. 2A and 2B, the attachment 260 is shown as having two parallel articulating arms 254 with two linkages each, and five revolute joints 256, which the linkages rotate about laterally. In certain embodiments, the attachment 260 further includes a distal revolute joint 258 at a distal end thereof to enable rotation of the surgical tool assembly 140 about a horizontal axis.

In some embodiments, the eye tracking system 150 is also connected to the forehead support 175. For example, the eye tracking system 150 may be connected to the surgical tool assembly 140 or to the attachment 260 through, e.g., a rigid link or articulating arms, or may be connected to the forehead support 175 through a separate structure. In other embodiments, some or all of the eye tracking system 150 is supported separately from the support system 165. For example, one or more sensors of the eye tracking system 150 may be implemented as “scene” sensors that are mounted on a static structure within the environment. In another example, one or more sensors of the eye tracking system 150 may be mounted on the end effector of the surgical tool 142.

In order to support the weight of the surgical tool assembly 140 and/or the eye tracking system 150, the forehead support 175 is further coupled to the counterbalancing and passive articulating arm 162 supported by the base 170. In certain embodiments, the articulating arm 162 includes a Selective Compliance Articulated Robot Arm (SCARA) mechanism 262 to allow passive, lockable horizontal movement of the forehead support 175 and the surgical tool assembly 140 attached thereto, as well as a four-bar parallelogram mechanism 270 to enable passive, lockable vertical movement thereof. For example, as shown in FIGS. 2A and 2B, the SCARA mechanism 262 is formed by at least two links 264 and at least three revolute joints 266 having vertical axes to create passive motion parallel to a floor plane of the operating room. The four-bar parallelogram mechanism 270 is formed by four bars 272 and four revolute joints 274 having horizontal axes to create passive vertical motion perpendicular to the floor plane. The four-bar parallelogram mechanism 270 is further counterbalanced by a spring 276, such as an air spring, constant force spring, or the like, which enables locking of the four-bar parallelogram mechanism 270. Together, the SCARA mechanism 262 and the four-bar parallelogram mechanism 270 provide an adjustable counterbalancing mechanism to account for the weight of the surgical tool assembly 140, which is mounted to and moves with the patient’s head 106 during a surgical procedure for increased patient safety in the context of patient head movement. Note that although the passive articulating arm 162 is shown as having the SCARA mechanism 262 at a distal end thereof and the four-bar parallelogram mechanism 270 at a proximal end thereof, the passive articulating arm 162 may include the two mechanisms in any order or arrangement as desired.

FIG. 2C illustrates an example schematic bottom-up view of the surgical tool assembly 140 and eye tracking system 150 of FIGS. 2A and 2B, according to one or more embodiments.

In some embodiments, the eye tracking system 150 comprises a housing 202 within which sensors of the eye tracking system 150 are at least partly disposed. As shown, the eye tracking system 150 comprises a stereo vision module 210 comprising two visual sensors 212-L, 212-R, an OCT sensor 215, a polarization sensor 220, and a ToF sensor 152 comprising an emitter 222 and a receiver 224. The housing 202 defines a lower surface 205 at which the one or more ToF sensors 152, the two visual sensors 212-L, 212-R, the OCT sensor 215, and the polarization sensor 220 are disposed. For example, the OCT sensor 215 may include a light source, beamsplitter, reference mirror, etc. disposed in the housing 202, where the beamsplitter is aligned with an aperture 216 formed through the lower surface 205. Other compositions and arrangements of sensors are also contemplated, such as multiple ToF sensors 152.

The housing 202 is connected to the surgical tool assembly 140 through a link 225. Although the link 225 is shown as rigid, alternate implementations may include articulating arms or other actuatable mechanisms that permit relative movement between the surgical tool assembly 140 and the housing 202.

FIG. 3 illustrates an example perspective view of the surgical tool assembly 140 of the surgical system 100 of FIG. 1, according to one or more embodiments. The surgical tool assembly 140 is configured to be mounted over the patient’s eye 108 and manipulate and/or operate a surgical tool 142 directly or indirectly attached thereto. In the embodiment of FIG. 3, the surgical tool 142 is coupled to a distal end of a tool shaft 318, which, in certain embodiments, is a device holder or sleeve.

As depicted, the tool shaft 318 is movably coupled to two sets 302a, 302b of three radially-extending and linearly-actuating actuator links 303 that act as the drive train for the surgical tool assembly 140. Actuation of the actuator links 303 results in manipulation of the tool shaft 318 and thus, the surgical tool 142, and is determined by the control signal received from the operator device. The two sets of actuator links 303 include a first proximal set 302a and a second distal set 302b having parallel kinematics, thus enabling the two sets 302a, 302b to linearly move in concert (i.e., synchronously) to manipulate the tool shaft 318 and thus, the surgical tool 142, in response to control signals from the operator device. Utilization of a parallel and closed loop kinematic chain for the two sets 302a, 302b of actuator links 303 enables decreased structural weight and increased precision, stability, link rigidity, and acceleration, as compared to a single articulating arm equipped with serial kinematics. The parallel kinematic design of the surgical tool assembly 140 further enables differential drive of the two sets 302a, 302b of actuator links 303, thus providing greater maneuverability of the tool shaft 318 and surgical tool 142 while facilitating the averaging of joint error in the parallel link structure.

Each set 302a, 302b of actuator links 303 may be coupled to the tool shaft 318 at distal ends 312 of the actuator links 303 by a coupling ring 304 such that the actuator links 303 of each set 302a, 302b are attached to the tool shaft 318 on a single plane X1 or X2. The planes X1 and X2 are located at a proximal end 324 and a distal end 326 of the tool shaft 318, respectively. Accordingly, the embodiment of FIG. 3 may be described as a dual parallel tripod surgical tool assembly 140, having two sets 302a, 302b of three actuator links 303 (e.g., “three plus three”) extending radially outward from the tool shaft 318 at two different horizontal planes, thus forming two tripods of actuator links 303. The actuator links 303 may be radially spaced apart from adjacent actuator links 303 of the same set 302 by an angle of about 120° relative to the tool shaft 318.

Note that although three actuator links 303 are depicted in each set 302a, 302b in FIG. 3, it is further contemplated that a set may include other quantities of actuator links 303. For example, one or each of sets 302a, 302b may comprise four or more actuator links 303. In examples where a set includes more than three actuator links 303, a radial spacing between each actuator link 303 may be less than about 120°. Furthermore, although the actuator links 303 are described above as being coupled to the coupling rings 304, the actuator links 303 may be directly coupled to the tool shaft 318 or the surgical tool 142 via spherical joints without the utilization of a coupling ring.

In certain embodiments, proximal ends 314 of the actuator links 303 attach to a support frame 306 disposed radially outward of the tool shaft 318 and/or surgical tool 142. The proximal ends 314 couple to the support frame at attachment points located on different horizontal planes from the attachment points of the distal ends 312 with the coupling rings 304 or tool shaft 318 and/or surgical tool 142. Accordingly, the actuator links 303 may be described as being vertically angled (e.g., non-parallel with horizontal planes X1 and X2 or vertical axis Y of the surgical tool assembly 140). The support frame 306 may include any suitable structure to support the quantity of actuator links 303 utilized for the surgical tool assembly 140. In the embodiment of FIG. 3, the support frame 306 includes two ring-like bases 308 and three support columns 309 extending therebetween, which may be parallel to the vertical axis Y of the surgical tool assembly 140. Note that although two bases 308 and three support columns 309 are described, the surgical tool assembly 140 may include more or less bases and columns having any desired morphologies. The support columns 309 and/or bases 308 act as anchoring points for the actuator links 303, which may be coupled to the support columns 309 and/or bases 308 by any suitable type of spherical joints 315 enabling at least 3-DOF rotational movement. For example, the spherical joints 315 may have a ball-and-socket design, similar to that of the human hip joint, allowing free rotation of the actuator links 303 in two planes, while also preventing translation in any direction. In another example, the spherical joints 315 are gimbal-type spherical joints.

Similarly, the distal ends 312 of the actuator links 303 may also be coupled to the coupling rings 304, tool shaft 318, or surgical tool 142 by a spherical joint 316. The utilization of two spherical joints 315, 316 at opposing ends of the actuator links 303 enables movement of the surgical tool 142 in all three planes. Thus, the actuator links 303 may provide x, y, and z transitional movement as well as pitch and yaw rotational movement for the surgical tool 142, enabling up to 6-DOF of mobility for the surgical tool 142 (and up to 7-DOF when utilized with a rotary actuator coupled to the surgical tool 142, described below).

In some embodiments, the surgical tool assembly 140 further includes a rotary actuator (not shown) to provide 360° rotational movement of the tool shaft 318 and/or the surgical tool 142, thus enabling redundant 7-DOF tool roll of the surgical tool 142. In certain embodiments, the rotary actuator is coupled to or disposed within one of the one or more coupling rings 304 or the tool shaft 318, and thus may directly rotate the surgical tool 142. In other embodiments, the rotary actuator is coupled to the ring-like bases 308, enabling rotation of the support columns 309 and ultimately, the surgical tool 142. The rotary actuator may include any suitable type of rotary mechanism, including a zero-backlash piston driven rack and pinion, a single or dual rotary vane actuator, and the like. In some embodiments, the surgical tool assembly 140 also optionally includes a torque transducer or torque sensor coupled to or disposed within the coupling rings 304 and/or the tool shaft 318 for torque feedback.

The actuator links 303 of the surgical tool assembly 140 utilize a direct drive system with commutated linear motors 322 having electromagnetic brakes to manipulate the surgical tool 142. Thus, in some implementations, the actuator links 303 are linearly-actuating links having one or more direct drive actuators connected thereto. Utilization of commutated linear motors over more conventional motors may eliminate mechanical components that can introduce backlash or compliance and degrade positioning accuracy and repeatability, while also reducing load inertia and enabling more dynamic moves with less overshoot and oscillation. Further, commutated slotless linear motors facilitate smooth force control and high fidelity force feedback by enabling passive backdriving.

In certain embodiments, the motors 322 include 3-phase slotless brushless moving magnet linear motors with digital sine wave commutation and optional air bearings. For example, in certain embodiments, the motors 322 include slotless brushless DC (BLDC) linear motors. In such embodiments, the motors 322 may utilize a neodymium iron boron (NdFeB) magnet as a permanent magnet. Each motor 322 may be used in combination with a relative linear encoder (e.g., an optical or holographic linear encoder) for both commutation and control, and/or absolute linear encoders to remove the need for homing. The utilization of slotless brushless moving magnet linear motors provides several advantages over other types of motors (e.g., slotted motors), such as extremely small cogging torque (e.g., torque ripple). Thus, slotless brushless moving magnet linear motors enable more accurate driving with reduced vibration and noise during use thereof. Furthermore, the utilization of air bearings facilitates frictionless high-precision positioning with smooth, controlled velocity and high guiding accuracy. Together with the actuator links 303, the motors 322 form a direct drive system that enables the surgical system 100 to better perform force control, since geared and hydraulic drive systems may suffer from the effects of static and dynamic friction and/or backlash.

Note that, in certain embodiments, slotless brushless moving magnet linear motors, substantially similar to those of the surgical tool assembly 140, may also be utilized for the operator device. Furthermore, the rotary actuator (not shown) of the surgical tool assembly 140 may include a slotless BLDC-type moving magnet (NdFeB) motor to drive tool roll axis for the surgical tool 142.

As described above, the surgical tool assembly 140 is configured to indirectly mount to the head of a patient 102. Thus, in order to alleviate pressure on the patient’s head created by the weight of the surgical tool assembly 140 and, in particular, the drive motors 322, one or more components of the surgical tool assembly 140 may be formed of lightweight high modulus/density ratio materials. For example, in certain embodiments, the bases 308, support columns 309, coupling rings 304, and/or tool shaft 318 are formed of fiber reinforced engineering plastics, aluminum, Kevlar, carbon fiber, or the like in order to reduce weight applied to the patient’s head by the surgical system 100. In addition to utilizing lightweight materials for the surgical tool assembly 140, a counterbalancing support arm, such as the articulating arm 162, may be utilized to support the surgical tool assembly 140.

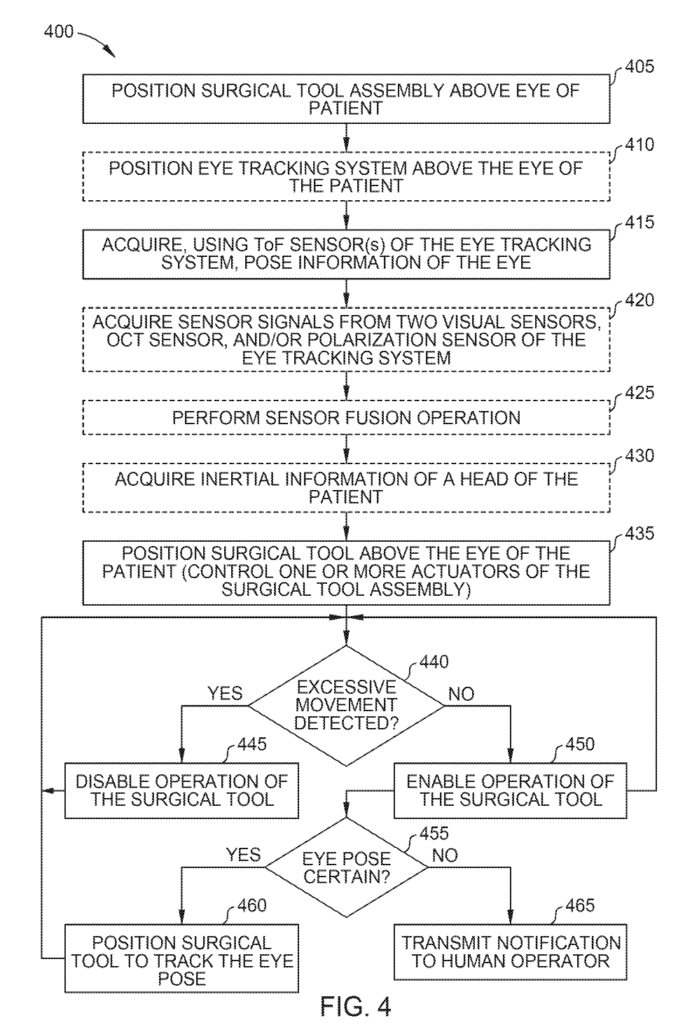

FIG. 4 illustrates an example method 400 of operating a surgical tool, according to one or more embodiments. The method 400 may be used in conjunction with other embodiments, e.g., being performed using the surgical tool control service 122 and/or the sensor fusion service 124 of FIG. 1.

The method 400 begins at block 405, where the surgical tool assembly 140 is positioned above the eye 108 of the patient 102. In some embodiments, positioning the surgical tool assembly 140 is performed manually, e.g., by manipulating the articulating arm 162 and/or the four-bar parallelogram mechanism 270 of the support system 165. In other embodiments, the surgical tool control service 122 may transmit control signals to operate one or more actuators and position components of the support system 165.

At an optional block 410, the eye tracking system 150 is positioned above the eye 108 of the patient 102. In some embodiments, positioning the eye tracking system 150 is performed manually, e.g., by manipulating components of the support system 165 (or of a separate support system). In other embodiments, the surgical tool control service 122 may transmit control signals to operate one or more actuators and position the components of the support system 165 or of the separate support system. In some embodiments, positioning the eye tracking system 150 may overlap in time with positioning the surgical tool assembly 140, e.g., when the surgical tool assembly 140 and the eye tracking system 150 are attached to each other.

At block 415, the surgical tool control service 122 acquires pose information of the eye 108 using one or more ToF sensors 152 of the eye tracking system 150. In some embodiments, the surgical tool control service 122 (at optional block 420) further acquires sensor signals from one or more additional sensors of the eye tracking system 150, such as the two visual sensors 212-L, 212-R, the OCT sensor 215, and/or the polarization sensor 220. In some embodiments, the sensor fusion service 124 (at optional block 425) performs a sensor fusion operation using the sensor signals received from at least one of: the one or more ToF sensors 152, the two visual sensors 212-L, 212-R, the OCT sensor 215, and the polarization sensor 220. In some embodiments, the sensor fusion service 124 implements a Kalman filter to perform the sensor fusion operation. Other types of sensor fusion operations are also contemplated in addition to, or alternate to, implementing the Kalman filter.

In some embodiments, the surgical tool control service 122 (at optional block 430) acquires inertial information of the head 106 of the patient 102. In some embodiments, the inertial information is received from an IMU of the head tracking system 155 disposed on the head 106 of the patient 102.

At block 435, the surgical tool control service 122 positions the surgical tool 142 above the eye 108 of the patient 102. In some embodiments, positioning the surgical tool 142 comprises controlling one or more actuators 144 of the surgical tool assembly 140 to position the surgical tool 142 relative to the eye 108. Controlling the one or more actuators 144 may be based on the pose information acquired by the one or more ToF sensors 152, the sensor fusion operation, and/or the inertial information acquired by the head tracking system 155.

At block 440, the surgical tool control service 122 determines, using the pose information acquired by the eye tracking system 150, whether excessive movement of the eye 108 is detected.

In some embodiments, excessive movement is detected when the movement of the eye 108 is greater than a threshold value. The threshold value may be a value of any of a displacement, velocity, or acceleration of the eye 108, which may be linear or angular.

In some embodiments, the threshold value depends on the surgical tool 142 and/or the surgical procedure being performed. In some embodiments, the surgical tool control service 122 may estimate a risk value representing a risk of damage to the eye 108 given the combination of the surgical tool 142 and the surgical procedure (and which may further be based on historical motion of the patient’s eye 108), and the risk value is used to determine the threshold value. For example, the surgical tool control service 122 may access a table (e.g., included in the surgical procedure information 126) storing a plurality of predefined risk values for particular combinations of the surgical tool 142 and the surgical procedure, and may access patient information (e.g., included in the surgical procedure information 126) to adjust the risk value based on the patient’s historical motion. In some cases, the historical motion of the patient 102 may be characterized (e.g., an average displacement of the eye 108, a maximum displacement, a frequency of motions greater than a threshold amount, and so forth), and the risk value may be increased for characteristics indicating a likelihood of frequent and/or more severe motion by the patient 102.

When excessive movement is detected (“YES”), flow proceeds from block 440 to block 445, where the surgical tool control service 122 disables operation of the surgical tool 142. In some embodiments, disabling operation of the surgical tool 142 comprises removing operative power to the surgical tool 142, such as electrical power, hydraulic power, pneumatic power, optical power, and so forth. In some embodiments, disabling operation of the surgical tool 142 comprises changing an operational characteristic of the surgical tool 142 (e.g., selecting a more conservative operational setting).

In some embodiments, disabling operation of the surgical tool 142 comprises retracting the surgical tool 142 (or a component thereof) to increase the distance from the surgical tool 142 to the eye 108 of the patient 102. In some embodiments, disabling operation of the surgical tool 142 further comprises transmitting a notification (e.g., a visual and/or audio warning message) to the patient 102 and/or the human operator 110 to alert them to the excessive eye movement. Flow returns to block 440, where the surgical tool control service 122 determines whether excessive movement is detected.

When excessive movement is not detected (“NO”), flow proceeds from block 440 to block 450, where the surgical tool control service 122 enables operation of the surgical tool 142. In some embodiments, enabling operation of the surgical tool 142 comprises maintaining operative power to the surgical tool 142.

Flow proceeds from block 450 to block 455, where the surgical tool control service 122 determines whether the eye pose is certain. In some embodiments, the surgical tool control service 122 accesses a confidence value associated with the pose information acquired by the one or more ToF sensors 152, and determines that the eye pose is certain when the confidence value exceeds a threshold value. For example, the confidence value may be less when the view of the eye 108 is partly occluded, e.g., by surgical equipment arranged between the one or more ToF sensors 152 and the eye 108. In some embodiments, when the view of a first ToF sensor 152 is occluded, the surgical tool control service 122 acquires pose information of the eye 108 using a second ToF sensor 152, and determines whether the eye pose is certain using the pose information from the second ToF sensor 152 and/or the first ToF sensor 152 (e.g., the pose information corresponding to the greater confidence value, or a combination of the pose information from the first ToF sensor 152 and from the second ToF sensor 152).

When the eye pose is determined to be not certain (“NO”), flow proceeds from block 455 to block 465, where the surgical tool control service 122 transmits a notification (e.g., a visual and/or audio warning message) to the human operator 110 to alert them to the not certain eye pose.

When the eye pose is determined to be certain (“YES”), flow proceeds from block 455 to block 460, where the surgical tool control service 122 positions the surgical tool 142 to track the eye pose. Flow returns to block 440, where the surgical tool control service 122 determines whether excessive movement of the eye 108 is detected. In some embodiments, block 440 and/or block 455 may be performed periodically (or substantially continuously) by the surgical tool control service 122 during the surgical procedure.

In summary, embodiments of the present disclosure provide an eye tracking system that improves safety and efficiency of robotic manipulation systems for ophthalmic microsurgical procedures. Performing eye tracking using ToF sensors provides high-resolution, high-precision tracking that supports an improved responsive control of surgical tools to minimize risk of unnecessary trauma to a patient’s eye. This also improves the efficiency of such procedures, allows them to be performed while the patient remains awake.

Although vitreous surgery is discussed as an example of a surgical procedure benefiting from the described embodiments, the advantages of the surgical devices and systems described herein may benefit other surgical procedures as well.

While the foregoing is directed to embodiments of the present disclosure, other and further embodiments of the disclosure may be devised without departing from the basic scope thereof, and the scope thereof is determined by the claims that follow.

What is claimed is:

1. A surgical system comprising:
a robotic manipulator;
a surgical tool assembly connected to the robotic manipulator, the surgical tool assembly comprising an actuatable surgical tool, wherein the robotic manipulator is configured to position the surgical tool assembly above an eye of a patient;
an eye tracking system comprising one or more Time of Flight (ToF) sensors positioned above the eye, the one or more ToF sensors configured to acquire pose information of the eye; and
a controller communicatively connected to the surgical tool and to the eye tracking system, the controller configured to disable operation of the surgical tool responsive to excessive movement of the eye that is indicated by the pose information.

2. The surgical system of claim 1, wherein the controller is further configured to:
receive the pose information from the eye tracking system; and
control, based on the pose information, one or more actuators of the surgical tool assembly to position the surgical tool relative to the eye.

3. The surgical system of claim 2, further comprising:
a head tracking system comprising an inertial measurement unit (IMU) disposed on the head of the patient, the IMU configured to acquire inertial information of the head,
wherein controlling the one or more actuators is further based on the inertial information.

4. The surgical system of claim 1, wherein the one or more ToF sensors comprises an indirect ToF sensor.

5. The surgical system of claim 1, the eye tracking system further comprising:
two visual sensors;
an optical coherence tomography (OCT) sensor; and
a polarization sensor.

6. The surgical system of claim 5, wherein the controller is further configured to:
perform a sensor fusion operation using sensor signals received from at least one of: the one or more ToF sensors, the two visual sensors, the OCT sensor, and the polarization sensor; and
detect, from the sensor fusion operation, the excessive movement of the eye.

7. The surgical system of claim 6, wherein the controller implements a Kalman filter to perform the sensor fusion operation.

8. The surgical system of claim 5, the eye tracking system further comprising:
a housing defining a lower surface at which the one or more ToF sensors, the two visual sensors, the OCT sensor, and the polarization sensor are disposed.

9. The surgical system of claim 8, wherein the housing is connected to the robotic manipulator.

10. The surgical system of claim 1, further comprising:
a support system configured to mount to a head of the patient, and to support at least the surgical tool assembly.

11. The surgical system of claim 10, wherein the support system further supports the eye tracking system.

12. The surgical system of claim 10, the support system comprising:
a U-shaped forehead pad;
an adjustable head strap connected to the forehead pad; and
at least one adjustable arm extending from the forehead pad and configured to support the surgical tool over the eye of the patient.

13. The surgical system of claim 12, wherein the support system further comprises a counterbalancing articulating arm attached to the forehead pad, the counterbalancing articulating arm comprising:
a SCARA mechanism; and
a passive four-bar parallelogram mechanism counterbalanced by a spring.

14. The surgical system of claim 1, further comprising:
a support frame;
a plurality of linearly-actuating links connected radially to the support frame and connected to the surgical tool; and
one or more direct drive actuators connected to each of the plurality of linearly-actuating links.

15. The surgical system of claim 1, further comprising:
an operator device communicatively connected to the controller,
wherein the controller is further configured to:
receive operator pose information from the operator device responsive to manipulation thereof by a human operator; and
control the one or more actuators of the surgical tool assembly based on the operator pose information.
